(19) United States
(12) Patent Application Publication
(10) Pub. No.: US 2012/0269913 A1
(43) Pub. Date: Oct. 25, 2012

(54) CRUDE CAFFEINE COMPLEX, IMPROVED FOOD PRODUCTS USING THE CRUDE CAFFEINE COMPLEX, AND METHODS OF USE THEREOF

(76) Inventors: Yi-Fang Chu, Glenview, IL (US); Jimbay P. Loh, Green Oaks, IL (US); Peter H. Brown, Glenview, IL (US); Claire E. Macaulay, Witney Oxfordshire (GB); Barbara J. Lyle, Deerfield, IL (US)

(21) Appl. No.: 13/384,884

(22) PCT Filed: Jul. 20, 2010
(86) PCT No.: PCT/US10/42611

§ 371 Date: Apr. 2, 2012

Related U.S. Application Data
(60) Provisional application No. 61/227,216, filed on Jul. 21, 2009.

(51) Int. Cl. A61K 36/74; A61P 3/08; A61P 29/00

(52) U.S. Cl. 424/769

(57) ABSTRACT

An improved food product comprising a comestible and a crude caffeine complex as a functional additive is provided. The crude caffeine complex includes a blend of caffeine and coffee-based biologically active compounds. The additive demonstrates an ability to stimulate glucose uptake into cells, provide antioxidant benefits, and anti-inflammatory activities. A method of using the crude caffeine complex and incorporating the crude caffeine complex in comestibles is also provided.

### CROSS-REFERENCE TO RELATED APPLICATIONS

This application claims the benefit of U.S. Provisional Application No. 61/227,216, filed Jul. 21, 2009, which is incorporated herein by reference.

### FIELD

The field relates to a crude caffeine complex, improved food products using the crude caffeine complex, and methods of use thereof.

### BACKGROUND

As consumers become more health conscious, they often desire more foods with health-functional benefits to incorporate into their diet. In some cases, a diet can be helpful in minimizing or controlling certain health related issues.

For example, diabetes occurs when the body does not produce enough insulin resulting in high blood glucose levels. In some cases, blood glucose can be maintained within normal levels by monitoring diet along with the type and selection of foods consumed. In other cases, blood glucose can be controlled through administering additional insulin, which facilitates or stimulates the uptake of glucose by cells. In some instances, however, diabetics can become resistant to insulin and the body no longer responds to its presence resulting in a more difficult time in controlling their blood glucose levels. Thus, there is a desire for additional ways to achieve health functional benefits from foods and diet, such as alternative ways to control blood glucose.

Caffeine or 1,3,7-trimethylxanthine is widely consumed in coffee and other beverages. It occurs naturally in the beans, leaves, and fruits of some plants, and is most commonly associated as occurring naturally in coffee beans and tea leaves. In other cases, pure caffeine, which is obtained through a decaffeination process and purified to isolate the caffeine is used as an additive to various beverages, foods, and drugs. The residue from the purification process is typically discarded as waste.

### SEGUE

Crude caffeine is a major byproduct of green coffee bean decaffeination. Decaffeinated coffee accounts for about 10% of overall coffee consumption; thus thousands of tons of crude caffeine are produced annually. In addition to caffeine, green coffee beans contain a wide variety of bioactive phytochemicals, including chlorogenic acids and coffee oils. Some of the non-caffeine phytochemicals are inevitably removed during the process of decaffeination.

Crude caffeine is the starting material for manufacturing pure caffeine, which is used in beverages, foods, and medicines; the non-caffeine residues are typically discarded as waste. In light of the many studies that have demonstrated health benefits of coffee phytochemicals, we postulated that crude caffeine may also exert beneficial effects due to the presence of these non-caffeine components.

### SUMMARY

In short, we identified multiple bioactivities in crude caffeine: strong antioxidant capacity, ability to stimulate glucose uptake in human fat and muscle cells, and inhibitory effects against the inflammatory enzyme COX-2. These activities were conferred by bioactive compounds that co-purified with caffeine during the decaffeination process.

In one aspect, improved comestibles such as foods, food products, and beverages are described that include an effective amount of a functional additive in the form of a crude caffeine complex including a blend of caffeine and effective amounts of bioactive compounds obtained from a coffee base. It has been discovered that this crude caffeine complex is effective to stimulate glucose uptake by cells, provide antioxidant properties, and/or anti-inflammatory benefits. While pure caffeine is commonly used in a number of products from drugs to caffeinated beverages, it has been discovered that pure caffeine (as opposed to the crude caffeine complexes herein) does not exhibit the functional properties discovered in the crude caffeine complex. Indeed, the crude caffeine complexes herein also demonstrate a more pronounced functional effect with respect to glucose uptake, antioxidant properties, and anti-inflammatory benefits on a per gram basis than caffeinated coffee and tea.

These unexpected benefits are believed to be the result of effective amounts of certain coffee-based bioactive compounds and/or a synergistic effect of the caffeine with the coffee-based bioactive compounds. These bioactive compounds are normally waste product from a decaffeination and purification process to achieve pure caffeine. Previously, the bioactives were discarded. While not wishing to be limited by theory, it is believed that the coffee-based bioactive compounds are obtained from the starting coffee bean and/or potentially sourced from the unroasted, green coffee bean. As such, crude caffeine obtained from other caffeine sources, such as tea, chocolate, and the like, would not be expected to demonstrate the same functional benefits as the crude caffeine complexes described herein because tea or chocolate-sourced caffeine does not have the coffee-based bioactive compounds. Again, while not wishing to be limited by theory, it is also believed that the functional benefits of the crude caffeine complex may be due to the concentration levels of and/or certain bioactive compounds in the complex relative to the caffeine and/or concentrations levels of the bioactive compounds in the absence of various lipids, saccharides, and other bulk components present in the starting coffee bean prior to decaffeination.

In one case, it has been discovered that effective dosages of the complex of crude caffeine and coffee-based bioactive compounds tend to exhibit the ability to control blood glucose levels similar to or better than the artificial use of insulin by stimulating glucose uptake in skeletal muscle cells or adipocytes cells. Such levels of glucose uptake in cells are not exhibited with the use of pure caffeine alone. As a result, it is anticipated that the methods and foods with the crude caffeine complexes herein could be used as a substitute for insulin in controlling blood glucose levels, especially for those diabetics that have become resistant to insulin. In other cases, it has been discovered that effective dosages of the complex of crude caffeine and coffee-based bioactive compounds may have the ability to be more effective in anti-inflammatory activity than aspirin and exhibit anti-oxidant properties.

As used herein, the crude caffeine complex includes a majority of unpurified caffeine, which is preferably obtained as the raw by-product of a typical decaffeination process that converts regular coffee to decaffeinated coffee. By one preferred approach, the crude caffeine complex includes a major amount of the caffeine and a minor amount of the coffee-based bioactive compounds. By another approach, the crude caffeine complex may include about 90% to about 95% un-purified caffeine and about 5% to about 10% biologically active compounds obtained from the coffee base or green coffee bean. In addition, the crude caffeine complex may also be substantially free of acids, sugars, proteins, and other bulk starting materials of coffee. By one approach, for example, the crude caffeine complex includes less than about 0.01 percent chlorogenic acids and their lactone derivatives, less than about 0.1 percent sugars, and less than about 0.01 percent proteins, which is intended to mean “substantially free of for purposes of this disclosure.

In another aspect, the improved food, food product, or beverage may include about 0.05 percent to about 25 percent of the crude caffeine complex, but such amount can vary depending on the particular food, functional benefit desired, and other factors. More specifically, about 0.1 to about 15 percent of the crude caffeine complex may be added to soft drinks, cookies, cheese, crackers, powdered beverages, roast and ground coffee, soluble coffee, and the like. Of course, these are but only a few examples of foods and beverages that the crude caffeine complex may be used with. In one particular approach, the crude caffeine complex is well suited for non-coffee-based applications. In yet other approaches, the crude caffeine complex may be taken separately from food, such as by injection, ingestion, or in a transdermal patch. The crude caffeine complex may be provided as a pill, capsule, chewing gum, film, and the like.

Alternatively, the coffee-based bioactives may be further refined and isolated from the caffeine and used independently of the caffeine. If the bioactives are used separately from the caffeine, it is expected that about 0.1 to about 15 percent may be mixed with various foods or beverages, such as soft drinks, cookies, cheese, crackers, powdered beverages, and/or roast and ground or soluble coffee, and the like.

In yet another aspect, methods of using the crude caffeine complex are provided. For example, methods are described for stimulating glucose uptake in cells, providing anti-oxidant properties, or anti-inflammatory activity in subjects or organisms needing such benefits through the use of or the consumption of effective amounts of the crude caffeine complex with or with out associated food and beverages. In one approach, the crude caffeine complex may be blended with a food or beverage or precursor ingredients of the food or beverage so that the complex is consumed, which is expected to provide the functional benefits. In another approach, it is believed that the crude caffeine complex may be consumed or taken directly to achieve such glucose uptake. In still another approach, methods of stimulating glucose uptake by skeletal muscle cells or adipocytes cells are provided by contacting such cells with effective dosage amounts of the crude caffeine complex.

In another aspect, the methods to obtain the crude caffeine complex and a food product obtained by such methods are also provided. By one approach, for example, an improved food product may be obtained by first decaffeinating coffee to yield a decaffeinated coffee product and a crude caffeine by-product having coffee-based bioactive compounds as a result of coelution with caffeine during the decaffeination. The crude caffeine by-product is then formed into the crude caffeine complex having about 90 to about 95 percent caffeine and about 5 to about 10 percent of the bioactive compounds, which may be blended with the comestibles, foods, or beverages to form the improved comestible, food, or beverage product. By one approach, the crude caffeine complex is obtained via a supercritical carbon dioxide decaffeination.

The crude caffeine complex may be directly added, sprayed, injected, blended, or otherwise incorporated into foods or other beverages or be added, sprayed, injected, blended, or incorporated into precursors or ingredients of the comestible, foods, or beverages. It is preferred that the crude caffeine complex, prior to incorporation into foods or beverages, may be stored at room temperatures and shielded from ambient light. As mentioned above, about 0.05 to about 25 percent of the crude caffeine complex may be blended to foods or beverages or to precursors of the foods or beverages, which is believed effective to achieve the functional benefits described above.

By yet another approach, the crude caffeine complex may also be further purified or refined to isolate or concentrate certain of the coffee-based bioactive compounds. If further purified, the biologically active compounds may be concentrated to certain levels or even separated from the caffeine to produce a coffee-based bioactive extract (which contains the coffee-based biologically active compounds and preferably less than about 1 percent caffeine). This bioactive extract may then be added to a food, beverage, or pharmaceutical product to provide increased glucose uptake, as well as antioxidant and anti-inflammatory activities.

### DRAWINGS

FIG. 1 is a flow chart of an exemplary crude caffeine extraction process having a comestible as an end product;

FIG. 2 is a flow chart of an exemplary crude caffeine extraction process having a drug as an end product;

FIG. 3 is a flow chart of an exemplary crude caffeine extraction process having a cosmetic as an end product;

FIG. 4 is a flow chart of an exemplary crude caffeine extraction process having a dietary supplement as an end product;

FIG. 5 is a flow chart of an exemplary crude caffeine extraction process having a biologic as an end product;

FIG. 6 is a graph of glucose uptake into human adipocytes cells or human skeletal muscle cells;

FIG. 7 is a graph of anti-inflammatory activity;

FIGS. 8A and 8B are chromatograms of a crude caffeine complex; and

FIG. 9 is a diagram of supercritical CO₂ extraction of caffeine from coffee.

### DETAILED DESCRIPTION

The term “food” means, as stated in the Federal Food, Drug, and Cosmetic Act, (1) an article used for food or drink for man or other animal, (2) chewing gum, and (3) an article used for a component of any such article.

The term “drug” means, as stated in the Federal Food, Drug, and Cosmetic Act, an article recognized in the official United States Pharmacopoeia, official Homoeopathic Pharmacopoeia of the United States, or official National Formulary, or any supplement to any of them, an article intended for use in the diagnosis, cure, mitigation, treatment, or prevention of disease in man or other animal, an article (other than food) intended to affect the structure or any function of the body of man or other animal, and an article intended for use as a component of any article just specified.

The term “cosmetic” means, as stated in the Federal Food, Drug, and Cosmetic Act, an article intended to be rubbed, poured, sprinkled, or sprayed on, introduced into, or otherwise applied to the human body or any part thereof for cleansing, beautifying, promoting attractiveness, or altering the appearance, and an article intended for use as a component of any such article.

The term “dietary supplement” means, as stated in the Federal Food, Drug, and Cosmetic Act, a product intended to supplement the diet that bears or contains one or more of the following dietary ingredients: a vitamin, a mineral, an herb or other botanical, an amino acid, a dietary substance for use by man to supplement the diet by increasing the total dietary intake, or a concentrate, metabolite, constituent, extract, or combination of any ingredient just described.

The term “biologic” means, as defined by the Food and Drug Administration (FDA), any virus, therapeutic serum, toxin, antitoxin, or analogous product applicable to the prevention, treatment or cure of diseases or injuries of man.

Food encompasses the following general food categories, as defined by the Food and Drug Administration (FDA): baked goods and baking mixes, including all ready-to-eat and ready-to-bake products, flours, and mixes requiring preparation before serving; beverages, alcoholic, including malt beverages, wines, distilled liquors, and cocktail mix; beverages and beverage bases, nonalcoholic, including only special or spiced teas, soft drinks, coffee substitutes, and fruit and vegetable flavored gelatin drinks; breakfast cereals, including ready-to-eat and instant and regular hot cereals; cheeses, including curd and whey cheeses, cream, natural, grating, processed, spread, dip, and miscellaneous cheeses; chewing gum, including all forms; coffee and tea, including regular, decaffeinated, and instant types; condiments and relishes, including plain seasoning sauces and spreads, olives, pickles, and relishes, but not spices or herbs; confections and frostings, including candy and flavored frostings, marshmallows, baking chocolate, and brown, lump, rock, maple, powdered, and raw sugars; dairy product analogs, including nondairy milk, frozen or liquid creamers, coffee whiteners, toppings, and other nondairy products; egg products, including liquid, frozen, or dried eggs, and egg dishes made therefrom, i.e., egg roll, egg foo young, egg salad, and frozen multicourse egg meals, but not fresh eggs; fats and oils, including margarine, dressings for salads, butter, salad oils, shortenings and cooking oils; fish products, including all prepared main dishes, salads, appetizers, frozen multicourse meals, and spreads containing fish, shellfish, and other aquatic animals, but not fresh fish; fresh eggs, including cooked eggs and egg dishes made only from fresh shell eggs; fresh fish, including only fresh and frozen fish, shellfish, and other aquatic animals; fresh fruits and fruit juices, including only raw fruits, citrus, melons, and berries, and home-prepared “ades” and punches made therefrom; fresh meats, including only fresh or home-frozen beef or veal, pork, lamb or mutton and home-prepared fresh meat-containing dishes, salads, appetizers, or sandwich spreads made therefrom; fresh poultry, including only fresh or home-frozen poultry and game birds and home-prepared fresh poultry-containing dishes, salads, appetizers, or sandwich spreads made therefrom; fresh vegetables, tomatoes, and potatoes, including only fresh and home-prepared vegetables; frozen dairy desserts and mixes, including ice cream, ice milks, sherbets, and other frozen dairy desserts and specialties; fruit and water ices, including all frozen fruit and water ices; gelatins, puddings, and fillings, including flavored gelatin desserts, puddings, custards, parfaits, pie fillings, and gelatin base salads; grain products and pastas, including macaroni and noodle products, rice dishes, and frozen multicourse meals, without meat or vegetables; gravies and sauces, including all meat sauces and gravies, and tomato, milk, buttery, and specialty sauces; hard candy and cough drops, including all hard type candies; herbs, seeds, spices, seasonings, blends, extracts, and flavorings, including all natural and artificial spices, blends, and flavors; jams and jellies, home-prepared, including only home-prepared jams, jellies, fruit butters, preserves, and sweet spreads; jams and jellies, commercial, including only commercially processed jams, jellies, fruit butters, preserves, and sweet spreads; meat products, including all meats and meat containing dishes, salads, appetizers, frozen multicourse meat meals, and sandwich ingredients prepared by commercial processing or using commercially processed meats with home preparation; milk, whole and skim, including only whole, lowfat, and skim fluid milks; milk products, including flavored milks and milk drinks, dry milks, toppings, snack dips, spreads, weight control milk beverages, and other milk origin products; nuts and nut products, including whole or shelled tree nuts, peanuts, coconut, and nut and peanut spreads; plant protein products, including the National Academy of Sciences/National Research Council “reconstituted vegetable protein” category, and meat, poultry, and fish substitutes, analogs, and extender products made from plant proteins; poultry products, including all poultry and poultry-containing dishes, salads, appetizers, frozen multicourse poultry meals, and sandwich ingredients prepared by commercial processing or using commercially processed poultry with home preparation; processed fruits and fruit juices, including all commercially processed fruits, citrus, berries, and mixtures; salads, juices and juice punches, concentrates, dilutions, “ades”, and drink substitutes made therefrom; processed vegetables and vegetable juices, including all commercially processed vegetables, vegetable dishes, frozen multicourse vegetable meals, and vegetable juices and blends; snack foods, including chips, pretzels, and other novelty snacks; soft candy, including candy bars, chocolates, fudge, mints, and other chewy or nougat candies; soups, home-prepared, including meat, fish, poultry, vegetable, and combination home-prepared soups; soups and soup mixes, including commercially prepared meat, fish, poultry, vegetable, and combination soups and soup mixes; sugar, white, granulated, including only white granulated sugar; sugar substitutes, including granulated, liquid, and tablet sugar substitutes; and sweet sauces, toppings, and syrups, including chocolate, berry, fruit, corn syrup, and maple sweet sauces and toppings.

In one aspect, complexes of crude caffeine with effective amounts of certain coffee-based bioactive compounds are provided as well as improved comestibles such as foods, food products, and beverages incorporating the crude caffeine complexes. It has been discovered that these complexes provide unique functional benefits not found in pure caffeine. For instance, it has been discovered that the crude caffeine complex demonstrates the ability to stimulate glucose uptake by cells, provide antioxidant properties, and/or anti-inflammatory benefits. Pure caffeine, on the other hand, which is almost 99 percent pure caffeine and substantially free of the bioactive compounds, does not exhibit these functional properties. The crude caffeine complexes are particularly suited for non-coffee-based food applications as an ingredient thereof, but the complexes may also be combined with roast and ground and soluble coffee products.

In other aspects, methods of using the crude caffeine complex are provided. By one approach, methods of stimulating glucose uptake in cells, such as human skeletal muscle cells or human adipocytes cells, are provided by contacting the cells with the crude caffeine complex. In other approaches, methods of reducing blood glucose in a subject or organism that has elevated blood glucose levels are provided wherein the subject or organism is contacted by or provided with the crude caffeine complex.

As used herein, the crude caffeine complex preferably includes a majority of caffeine, which can be obtained as the raw and unpurified by-product of a typical decaffeination process that converts coffee to decaffeinated coffee. The crude caffeine is isolated prior to the caffeine being purified to pure caffeine. The crude caffeine complex includes effective amounts of certain coffee-based bioactive compounds that are co-eluted with the caffeine during the decaffeination process. By one approach, the crude caffeine complex includes a major amount of the caffeine and a minor amount of the coffee-based bioactive compounds. For example, the crude caffeine complex may include about 90 to about 95 percent caffeine and about 5 to about 10 percent biologically active compounds obtained from the coffee base and/or unroasted or green coffee bean. In addition, the crude caffeine complex may also be substantially free of acids, such as chlorogenic acids, sugars, proteins, and catechins. As used herein, substantially free is intended to mean less than about 0.1 percent, and preferably, less than about 0.01 percent. By one approach, for example, the crude caffeine complex includes less than about 0.01 percent chlorogenic acids and their lactone derivatives, less than about 0.1 percent sugars, and less than about 0.01 percent proteins. These bioactive compounds are normally waste byproduct from a decaffeination and purification process to achieve pure caffeine. Previously, the bioactives were discarded. Thus, the complexes herein provide a new use for a previously discarded and wasted product.

It is believed that the crude caffeine complex is particularly effective as a functional ingredient to be used in various food products, such as in non-coffee applications like beverages, soft drinks, cookies, cheese, crackers, powdered beverages, and the like. The complexes may also be recombined with roast and ground coffee, soluble coffee, and liquid coffee beverages. In one aspect, therefore, foods, food products, and beverages can be provided that include an effective amount of the crude caffeine complex as an additional ingredient sufficient to provide the functional benefits described herein. The crude caffeine complex can be blended with the food as an ingredient thereof, or it can be blended with various precursors or ingredients of the comestible.

More specifically, it has been discovered that glucose uptake into cells, antioxidant properties, and anti-inflammatory activities are exhibited in laboratory tests by the crude caffeine complexes described herein, which include an unique complex or blend of crude caffeine along with effective amounts of certain coffee-based bioactive compounds. Pure caffeine does not exhibit these effects. While not wishing to be limited by theory, these unexpected benefits are believed to be a result of the coffee-based bioactive compounds and/or a combined synergistic benefit of the unpurified caffeine with the coffee-based bioactives. As used herein, bioactives or bioactive compounds refer to compounds that co-elute with caffeine during the decaffeination process and are effective either alone or in combination to provide the functional benefits described above. While not wishing to be limited by theory, it is believed that various combinations of these bioactive compounds may be responsible for each of the functional benefits described above.

Turning to more of the details, it has been discovered that in some instances a complex of crude caffeine and coffee-based bioactives exhibit the ability to control blood glucose levels similar to or better than the artificial use of insulin by stimulating glucose uptake into cells. For instance, laboratory testing has shown that a dosage of about 0.01 mg/mL of the crude caffeine complexes herein demonstrate a similar ability to stimulate glucose uptake into human skeletal muscle cells, but demonstrate an increased ability to stimulate glucose uptake into human adipocytes or fat cells as determined by glucose uptake primary cell testing. By one approach, it is expected that about 0.001 to about 0.1 mg/mL of the crude caffeine complex is expected to be as effective as 100 nanomolar of insulin in stimulating glucose uptake in cells. As a result, it is anticipated that the methods and foods with the crude caffeine complexes herein can be used as a substitute for insulin in controlling blood glucose levels, especially for those diabetics that have become resistant to insulin.

Such levels of glucose uptake in cells are not exhibited with the use of pure caffeine alone. This result is unexpected because it was believed that crude caffeine would exhibit a decreased effect (relative to pure caffeine) due to its lower levels of caffeine. As crude caffeine contains about 90 to about 95 percent caffeine and pure caffeine is about 99 percent caffeine, it was expected that the crude caffeine complex would exhibit a functional benefit about 9 to about 4 percent lower than pure caffeine. However, explained in more detail below, the testing demonstrated that the crude caffeine complex, with lower amounts of caffeine (but higher relative levels of the bioactive compounds), actually shows an increased glucose uptake effect compared to pure caffeine.

In other cases, it has been discovered in lab testing that a complex of caffeine and coffee-based bioactives can be more effective in anti-inflammatory activity than aspirin. It is generally understood that the enzyme cyclooxygenase-2 (“COX-2”) synthesizes prostaglandins, which can cause inflammation. Typically, inflammation can be controlled using either steroidal drugs or non-steroidal anti-inflammatory drugs (“NSAID”). Indeed, several NSAID COX-2 inhibitors are known. Common NSAID COX-2 inhibitors include aspirin and ibuprofen. It has been discovered through laboratory testing that the crude caffeine complexes herein demonstrate significantly more ability to inhibit COX-2 activity than aspirin. By one approach, a minimal dosage of the crude caffeine of about 0.02 mg/mL is as effective as aspirin at 0.19 mg/mL to inhibit about 50 percent of the tested enzymatic activity of COX-2.

In other cases, it has been discovered through laboratory testing that the crude caffeine complexes also demonstrate both hydrophilic and lipophilic anti-oxidant activities. Pure caffeine demonstrates little such activities.

As explained above, it is anticipated that the crude caffeine complex is particularly well suited as an ingredient in food products. Thus, by one approach, an improved food, food product, or beverage is provided herein that may include about 0.05 percent to about 25 percent of the crude caffeine complex as an effective amount to achieve the functional benefits described above, but such amount can vary depending on the particular food, functional benefit desired, and other factors. In one particular example, it is expected that about 0.1 to about 15 percent of the crude caffeine complex may be added to beverages, soft drinks, snacks, candies, gums, cookies, cheese, crackers, powdered beverages, and the like. Of course, this list of exemplary food is only but a few examples of where the complex may be used as a food additive. The crude caffeine complex may also be added to roast and ground coffee, soluble coffee, and the like. In yet other approaches, the crude caffeine complex may be taken separately from food, such as by injection, ingestion as a pill, capsule, used in a transdermal patch, and the like. To this end, the crude caffeine complex may be formed into pills, capsules, tablets, films, coatings, and other consumable forms.

The crude caffeine complex may be obtained from a decaffeination process of unroasted, green coffee beans. By one approach, the green coffee beans may be decaffeinated to yield a decaffeinated coffee product and a crude caffeine by-product. Undesired materials, such as activated carbon and the like, may optionally be removed from the product, and the crude caffeine may optionally be enriched as needed to isolate or concentrate particular bioactive compounds to form the final crude caffeine complex suitable as a food ingredient. By another approach, the crude caffeine by-product is then formed into the crude caffeine complex having about 90 to about 95 percent caffeine and about 5 to about 10 percent of the bioactive compounds. The crude caffeine complex may then be directly incorporated into foods or other beverages or, as mentioned above, be blended into precursors or ingredients of the foods or beverages. As mentioned, about 0.05 to about 25 percent of the crude caffeine complex may be blended to foods or beverages or to precursors of the foods or beverages to achieve the functional benefits described above.

By another approach, the crude caffeine complex may also be further purified or refined to isolate the coffee-based bioactive or enriched to concentrate certain bioactives responsible for the functional benefits described above. If further purified or enriched, the biologically active compounds may be separated from the caffeine to produce a pure caffeine extract and a bioactive extract (which contains the biologically active compounds) and a minor amount or no caffeine. Optionally, the purified coffee-based bioactives may be concentrated. This bioactive extract may then be added to a food, beverage, or pharmaceutical product to provide increased glucose uptake, as well as antioxidant and anti-inflammatory activities. If the purified bioactives are used separately from the crude caffeine, it is expected that about 0.1 to about 15 percent may be mixed with various foods or beverages, such as beverages, soft drinks, snacks, candies, gums, cookies, cheese, crackers, powders beverages, roast and ground coffee, soluble coffee, and the like. Again, this is only an exemplary list of foods believed to be suitable for the crude caffeine complex.

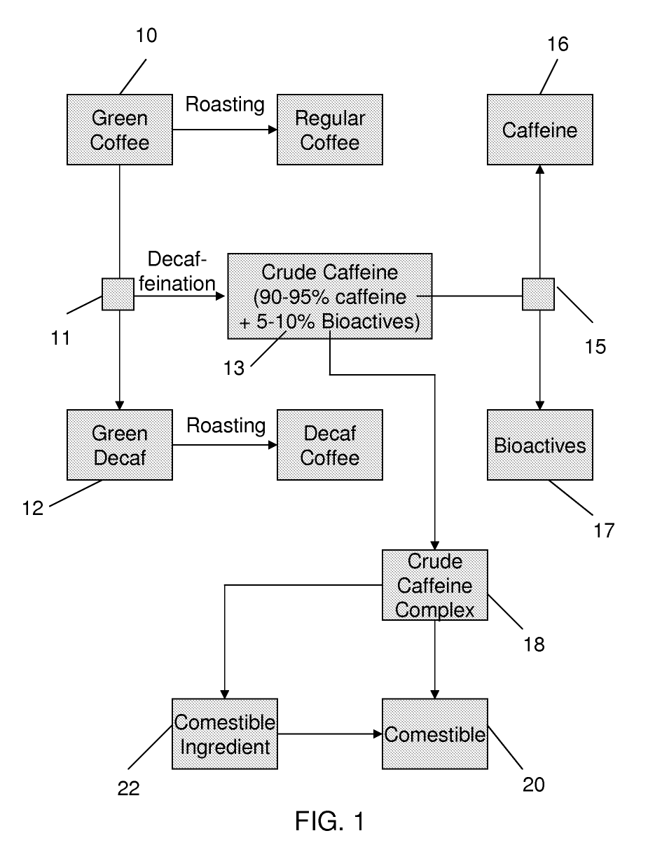

Turning to FIG. 1, an exemplary process to extract and form the crude caffeine complex is illustrated. For example, the starting source may be unroasted, green coffee beans 10 that are subjected to a decaffeination process 11 to produce decaffeinated coffee beans 12 and a crude caffeine extract 13 as a by-product. By one approach, the raw, green coffee beans are decaffeinated by supercritical carbon dioxide decaffeination process where carbon dioxide in liquid form is used to extract caffeine from coffee. The carbon dioxide may then be evaporated out of the crude caffeine complex. Often, the crude caffeine extract 13 is either disposed of as waste or further refined 15 to produce a pure caffeine extract 16. Optionally, the crude caffeine extract 13 may be further purified to form a substantially pure bioactive component 17 from the residue using the refining step 15. The crude caffeine complex 18 can be obtained from the by-product 13 straight from the decaffeination process, or the crude caffeine complex may be formed by concentrating, enriching, refining, or even blending desired amounts of the reside 17 (which is the isolated coffee-based bioactives) together in desired ratios with the caffeine. The crude caffeine complex 18 may then be added to various foods, beverage, food products, drugs, and other comestibles as desired. Alternatively, the crude caffeine complex may also be blended with various precursors of food and food products as explained further below. As further shown in FIG. 1, the improved food product or comestible 20 may be obtained by blending the crude caffeine complex 18 either directly into the comestible 20 or by first blending the crude caffeine complex 18 into an ingredient or precursor 22 of the comestible.

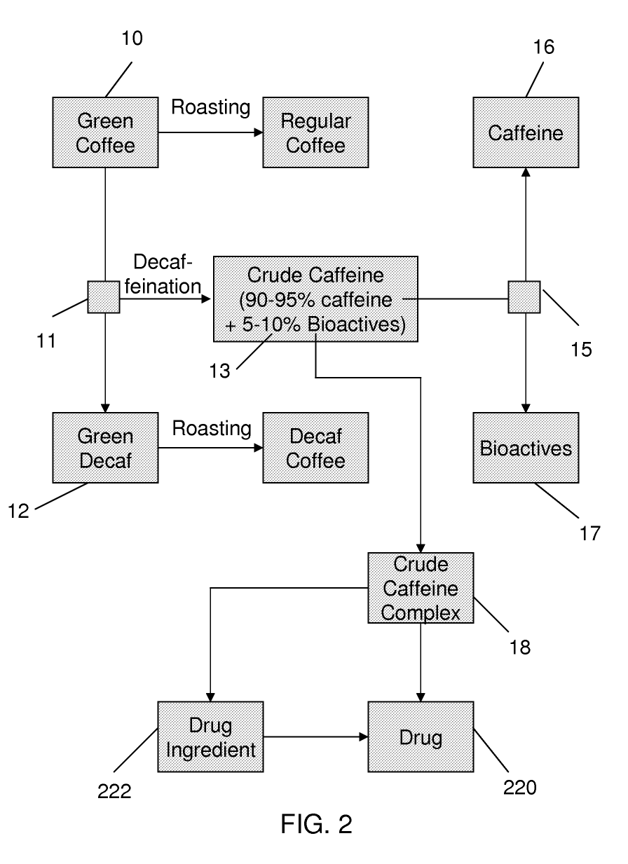

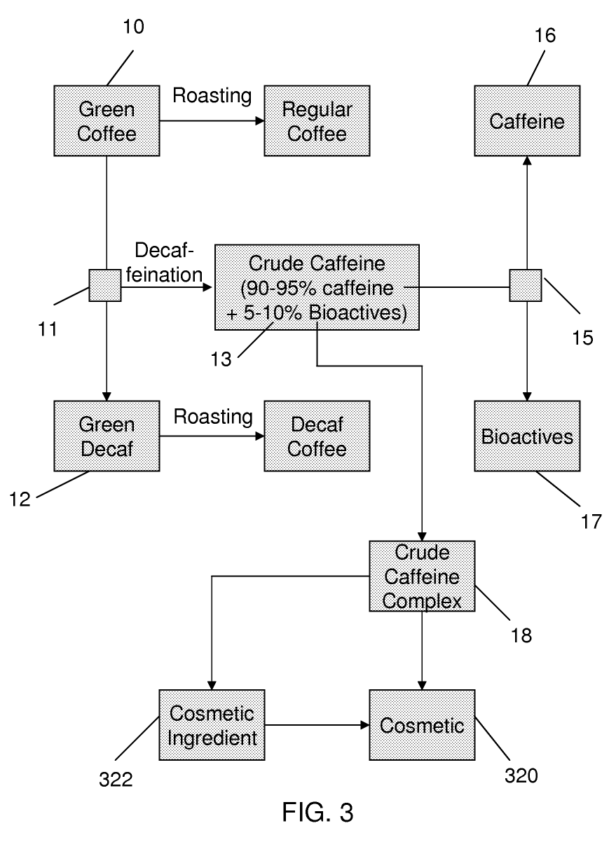

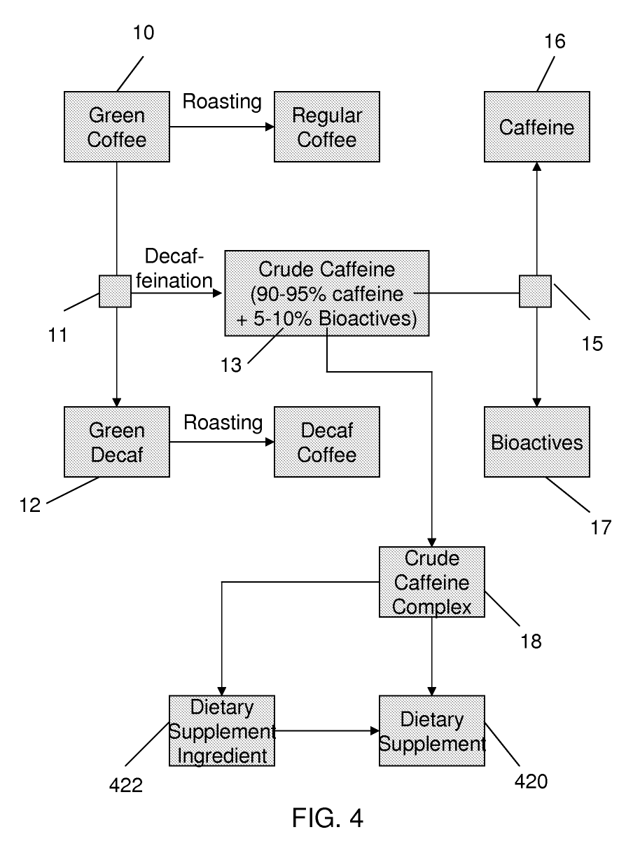

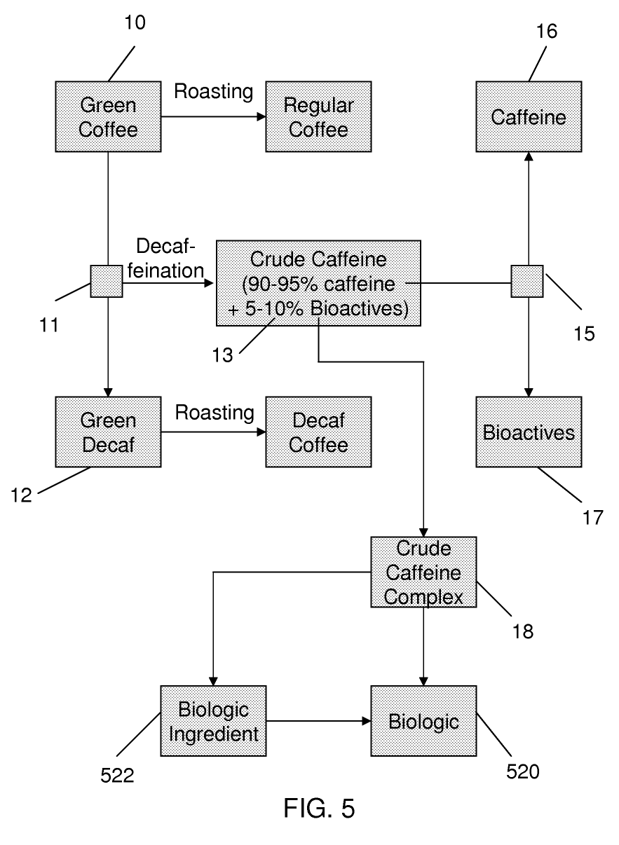

Similar to FIG. 1 being a flow chart of an exemplary crude caffeine extraction process having a comestible as an end product, FIG. 2 is a flow chart of an exemplary crude caffeine extraction process having a drug as an end product, FIG. 3 is a flow chart of an exemplary crude caffeine extraction process having a cosmetic as an end product, FIG. 4 is a flow chart of an exemplary crude caffeine extraction process having a dietary supplement as an end product, and FIG. 5 is a flow chart of an exemplary crude caffeine extraction process having a biologic as an end product.

As in FIG. 1, so in FIGS. 2 to 5, the starting source may be unroasted, green coffee beans 10 that are subjected to a decaffeination process 11 to produce decaffeinated coffee beans 12 and a crude caffeine extract 13 as a by-product. By one approach, the raw, green coffee beans are decaffeinated by supercritical carbon dioxide decaffeination process where carbon dioxide in liquid form is used to extract caffeine from coffee. The carbon dioxide may then be evaporated out of the crude caffeine complex. Often, the crude caffeine extract 13 is either disposed of as waste or further refined 15 to produce a pure caffeine extract 16. Optionally, the crude caffeine extract 13 may be further purified to form a substantially pure bioactive component 17 from the residue using the refining step 15. The crude caffeine complex 18 can be obtained from the by-product 13 straight from the decaffeination process, or the crude caffeine complex may be formed by concentrating, enriching, refining, or even blending desired amounts of the reside 17 (which is the isolated coffee-based bioactives) together in desired ratios with the caffeine. The crude caffeine complex 18 may then be added to various drugs (FIG. 2), cosmetics (FIG. 3), dietary supplements (FIG. 4), and biologics (FIG. 5), as desired. Alternatively, the crude caffeine complex may also be blended with various precursors of drugs (FIG. 2), cosmetics (FIG. 3), dietary supplements (FIG. 4), and biologics (FIG. 5). To wit, and as further shown in the figures, the improved drug 220 (FIG. 2), cosmetic 320 (FIG. 3), dietary supplement 420 (FIG. 4), and biologic 520 (FIG. 5) may be obtained by blending the crude caffeine complex 18 either directly into the drug 220 (FIG. 2), cosmetic 320 (FIG. 3), dietary supplement 420 (FIG. 4), and biologic 520 (FIG. 5), or by first blending the crude caffeine complex 18 into a drug ingredient or precursor 222 (FIG. 2), a cosmetic ingredient or precursor 322 (FIG. 3), a dietary supplement ingredient or precursor 422 (FIG. 4), and a biologic ingredient or precursor 522 (FIG. 5), of the respective drug (FIG. 2), cosmetic (FIG. 3), dietary supplement (FIG. 4), and biologic (FIG. 5).

In some approaches, the compositions of matter, whether food, drug, cosmetic, dietary supplement, or biologic, do not reproduce non-decaffeinated coffee beans or coffee, or produce regular coffee.

### EXAMPLES

All percentages are by weight unless otherwise indicated.

### Example 1

A crude caffeine extract was obtained by decaffeination of unroasted, green coffee beans by carbon dioxide under supercritical conditions (Maximus Coffee Group, Houston, Tex.). The crude caffeine extract was then analyzed to determine the crude caffeine contents. (Silliker, Inc., South Holland, Ill.) The assay results showed a caffeine concentration of about 94.8%. Thus, the extract had at most about 5.2 percent coffee-based bioactive compounds.

Another extract of crude caffeine produced by supercritical carbon dioxide (scCO2) decaffeination was evaluated. Table 1 shows the proximate composition of crude caffeine. The primary components were caffeine (95.95%), moisture (1.10%), and fat (1.04%); amounts of ash, fiber, protein, and sugar were negligible.

TABLE 1
Proximate composition of crude caffeine.

| Components | Mass (%) |
|---|---|
| Caffeine | 95.95 |
| Moisture | 1.10 |
| Fat | 1.04 |
| Ash | 0.1 |
| Total dietary fiber | <0.47 |
| Soluble dietary fiber | <0.47 |
| Insoluble dietary fiber | <0.47 |
| Protein | <0.01 |
| Fructose | <0.10 |
| Glucose | <0.10 |
| Sucrose | <0.10 |
| Lactose | <0.10 |
| Maltose | <0.10 |

Phenolic compounds are a major family of phytochemicals found in green coffee, contributing about 10% of the mass. Some of the phenolic compounds form complexes with caffeine and are therefore likely to be removed by decaffeination. We determined the presence of phenolics in the crude caffeine using the Folin-Ciocalteu assay, and found that the level of total phenolics was 10 mg CE/g, or approximately 1% of crude caffeine by mass.

### Example 2

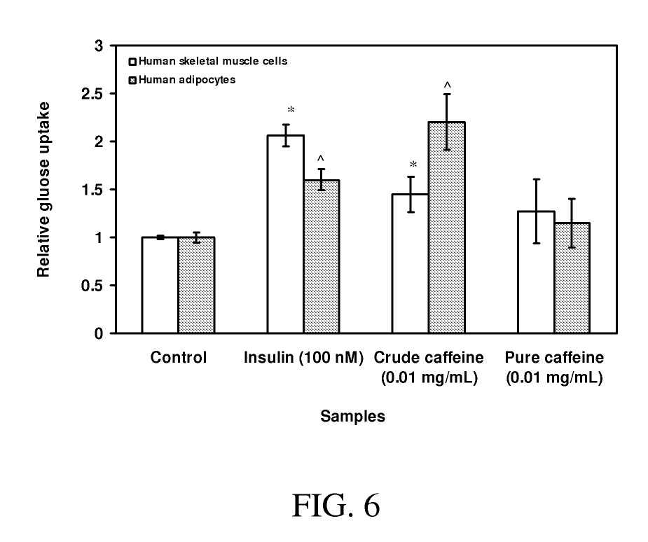

The effect of the crude caffeine extract of Example 1 on glucose uptake in both human adipocytes (fat) and skeletal muscle cells (SMC) was tested (Zen Bio Research, Triangle Park, N.C.). The same test was performed to evaluate the effect of pure caffeine (BASF, Florham Park, N.J.) on glucose uptake. It was originally believed that if the caffeine component contributed to the health effects, a correlation would exist such that the crude caffeine extract would affect health benefits, such as glucose uptake activity, about 10 to about 5 percent less than that demonstrated by pure caffeine because crude caffeine is about 90 to about 95 percent pure caffeine (that is, crude caffeine has about 5 to about 10 percent less caffeine). However, it was unexpectedly discovered that the crude caffeine complex (with less caffeine) actually exhibited improved glucose uptake over similar amounts of pure caffeine. Specifically and referring to FIG. 6, a dosage of about 0.01 mg/mL of crude caffeine complex increased glucose uptake in adipocytes cells by 120% (p<0.05) as compared to a control, while about 0.01 mg/mL of pure caffeine statistically showed no such effect. These results are graphically shown in FIG. 6 and compared to insulin. By one approach, the crude caffeine increases glucose uptake into adipocytes by at least 90% more than pure caffeine, as shown in FIG. 6.

In human skeletal muscle cells, a dosage of about 0.01 mg/mL of the crude caffeine extract increased glucose uptake by 45% (p<0.05) as compared to a control, while again, pure caffeine statistically showed no such effect. These results are also shown graphically in FIG. 6 and compared to insulin in FIG. 6.

It is believed that a dosage of crude caffeine from about 0.001 to about 0.1 mg/mL is more effective or at least no less effective than insulin for stimulating glucose uptake into cells. The insulin was tested at 100 nM (nanomolar), which is a standard dosage for glucose uptake in cell culture models.

In sum, FIG. 6 illustrates stimulation of glucose uptake in human skeletal muscle cells and adipocytes by insulin (100 nM), crude caffeine (0.01 mg/mL), and pure caffeine (0.01 mg/mL). Data were analyzed by one-way ANOVA followed by Tukey's post hoc test for multiple comparisons. In FIG. 6, results are expressed as mean±SD. The asterisk * denotes P<0.05 compared with human skeletal muscle cells; the carrot top A denotes P<0.05 compared with untreated human adipocytes.

In brief, we determined the effects of crude caffeine on glucose uptake in human skeletal muscle cells and adipocytes, using insulin as a positive control. In human skeletal muscle cells, 100 nM insulin stimulated a 2.06-fold increase in glucose uptake, and although less effective than insulin, 0.01 mg/mL crude caffeine increased glucose uptake significantly by 1.45 fold. In contrast, 0.01 mg/mL pure caffeine produced no significant effect. Surprisingly, in human adipocytes, crude caffeine stimulated glucose uptake more effectively than insulin: 0.01 mg/mL crude caffeine induced a 2.20-fold increase in glucose uptake, whereas 100 nM insulin induced a 1.6-fold increase. Pure caffeine (0.01 mg/mL) did not exert a significant effect on glucose uptake.

### Example 3

The crude caffeine complex of Example 1 was also tested for antioxidant activity and compared to the antioxidant activity of pure caffeine. Antioxidants may be physically classified by their solubility as either hydrophilic (water-soluble) or lipophilic (fat-soluble). Each type of antioxidant prevents oxidative damage in different parts of the body. Thus, to prevent as much oxidative damage as possible, it is desirable to have both types of antioxidants. We found that the ORAC_{hydro} was 329 μmol TE/g and the ORACₗᵢₚₒ was 129 μmol TE/g, whereas pure caffeine was associated with an extremely low ORAC_{hydro} (5 μmol TE/g) and ORACₗᵢₚₒ (0 μmol TE/g). It was thus discovered that crude caffeine is high in both hydrophilic and lipophilic antioxidant activities. Pure caffeine, on the other hand, showed little hydrophilic antioxidant activity, and no lipophilic antioxidant activity.

Another couple of extracts of crude caffeine produced by supercritical carbon dioxide (scCO2) decaffeination were evaluated. In the one, the ORAC_{hydro} was 145 μmol TE/g and the ORACₗᵢₚₒ was 66 μmol TE/g. In the other, the ORAC_{hydro} was 288 μmol TE/g and the ORACₗᵢₚₒ was 115 μmol TE/g.

By one approach, where crude caffeine is used as an additive, the hydrophilic antioxidant activity may have an oxygen radical absorbance capacity (ORAC) value of at least about 150 to about 300 μmole TE/g (micromoles of Trolox equivalents). By another approach, where crude caffeine is used as an additive, the lipophilic antioxidant activity may have an ORAC value of at least about 50 to about 100 μmole TE/g.

Using an ORAC assay adapted for the linoleate microemulsion system (Sim, W. L. S. et al., Journal of Agricultural and Food Chemistry 57: 3409-3414 (2009)), we determined the antioxidant activity of α-tocopherol (a form of vitamin E) combined with crude caffeine, ascorbic acid, and chlorogenic acid. Data were expressed as relative ORAC: a relative ORAC of 100% means that the antioxidant activity of the mixture equaled the sum of each component's antioxidant activity (additive effect), whereas a relative ORAC greater than 100% means that the antioxidant activity of the mixture was greater than the sum of each component's antioxidant activity (synergistic effect).

We found that crude caffeine and ascorbic acid produced an additive antioxidant effect in combination with α-tocopherol (relative ORAC of 100%). In contrast, chlorogenic acid produced a synergistic effect in combination with α-tocopherol (relative ORAC of 168%).

Our discovery that crude caffeine acts additively with α-tocopherol indicates the envisioned use of crude caffeine to substitute for the benefits of α-tocopherol-based dietary antioxidant supplements.

### Example 4

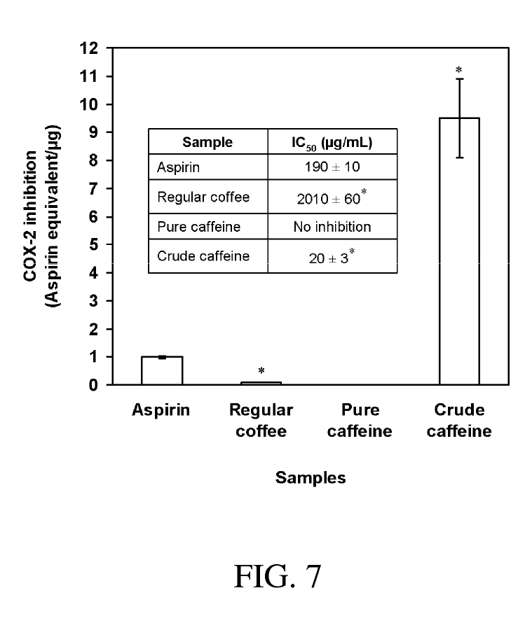

The ability of the crude caffeine extract of Example 1 to act as a COX-2 inhibitor was also tested and compared to pure caffeine and aspirin, a common NSAID anti-inflammatory agent. It was discovered that the crude caffeine complex demonstrated the ability to inhibit COX-2 activity greater than aspirin, whereas pure caffeine showed no such inhibitory activity. As discussed above, the ability to inhibit the COX-2 enzyme is understood as providing anti-inflammatory activities. These results are shown graphically in FIG. 7. The table inset shows values for IC₅₀ in mg/mL. IC₅₀ indicates the effective dose needed to achieve about 50 percent inhibition of the COX-2 enzyme. A lower number indicates that less compound is needed in solution to give the 50 percent inhibition level. As shown in the chart, aspirin has a value of about 0.19 mg/mL while crude caffeine has a value of about 0.02 mg/mL. Thus, substantially less crude caffeine than aspirin was needed in this test to achieve the 50% inhibition. Regular coffee, on the other hand, has a value of 2.01 mg/mL indicating that a much higher amount of pure caffeine is needed to achieve the same effect.

In sum, FIG. 7 illustrates inhibition of COX-2 by aspirin, regular coffee, caffeine, and crude caffeine. Data were analyzed by one-way ANOVA followed by Tukey's post hoc test for multiple comparisons. In FIG. 7, results are expressed as mean±SD. The asterisk * denotes P<0.05 compared with aspirin.

In brief, we evaluated the anti-inflammatory activity of crude caffeine by determining its effect on the inflammatory enzyme COX-2, using aspirin as a positive control. Crude caffeine inhibited COX-2 activity; the IC₅₀ value was 20 μg/mL, which was 9.5 times lower than that of aspirin (IC₅₀, 190 μg/mL), indicating that crude caffeine is a more potent COX-2 inhibitor than aspirin. In contrast, the activity of regular coffee (IC₅₀, 2010 μg/mL) was about 10 times lower than that of aspirin. Pure caffeine did not inhibit COX-2 activity.

### Example 5

The crude caffeine complex of Example 1 was further analyzed to partially determine some of the components of the coffee-based bioactives in the 5 to 10 percent minor component of the complex (Brunswick Labs, Norton, Mass.). The caffeine was separated from the bioactive extract by the following procedure: (1) dissolve 48.90 mg of crude caffeine in 15 mL deionized (DI) water and sonicated until the crude caffeine extract was dissolved; (2) add 30 mL of dichloromethane (CH₂Cl₂), shake, centrifuge and collect the CH₂Cl₂ layer (bottom layer); (3) repeat step 2 repeating 3-4 times; (4) add about 15 mL acetone into the water layer (bioactive extract), mix and sonicate for analysis.

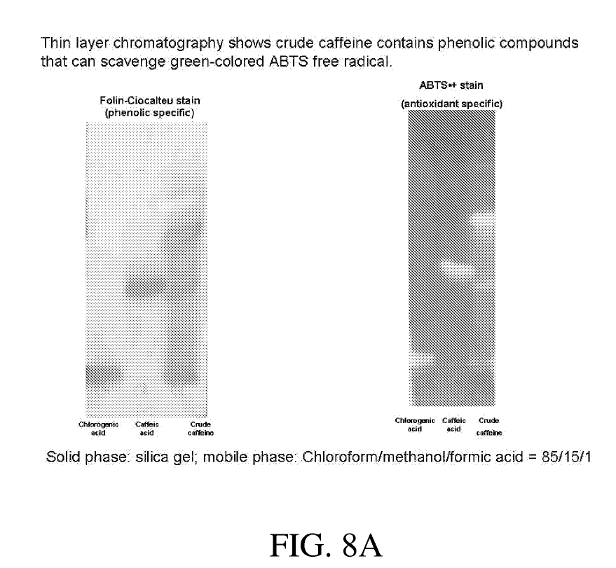

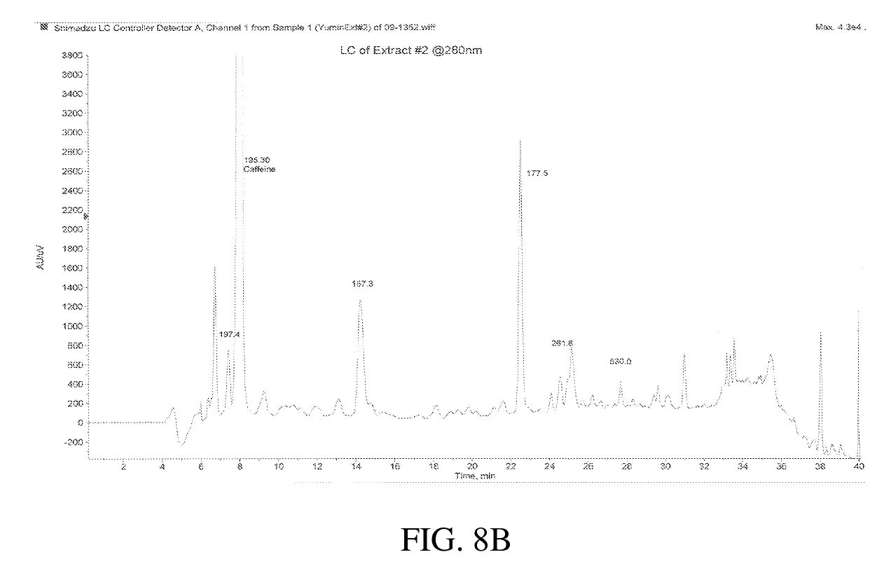

Analysis was then performed on the crude caffeine and the isolated bioactive extract using thin layer liquid chromatography, high performance liquid chromatography, and mass spectrometry to prepare chromatograms of the bioactive extract. Analysis charts of the extract are provided in FIGS. 8A and 8B. Based upon the charts of FIG. 8A and phenolic-specific Folin-Ciocalteu stains, it is believed that at least one component of the bioactives may be a phenolic acid compound.

The graph of FIG. 8B provides a mass spectrometry chart of the components in crude caffeine and shows the presence of non caffeine bioactive compounds by showing their mass spectroscopic fingerprints. As shown, caffeine has a peak with a molecular weight of 195.3 as labeled in the graph of FIG. 8B. In particular, the molecular weights of at least three potential bioactive compounds in the complex are shown at 197.4, 187.3, and 177.5. It is believed that these three compounds either individually or mixtures thereof may be major components of the bioactive composition of the crude complex. While not wishing to be limited by theory, it is believed that the content in the crude caffeine complex of these three major compounds individually may range from about 0 to about 20 percent each of the bioactive component. Without caffeine, the purified bioactive extract may be expected to contain between 0 and 100 percent of these three major compounds in a purified extract, individually, or mixtures thereof. The other peaks in FIG. 8B indicate other minor compounds also found in the crude caffeine.

To recap, thin layer chromatography (FIG. 8A) shows crude caffeine contains non-caffeine phenolic antioxidant by specific stains. The third stained band from the bottom in lane 3 of the left-hand gel was scraped from the chromatograph and was further analyzed by LC-MS.

To continue, LC-MS (FIG. 8B) shows the molecular weights of the finger print compounds contained in the scraped band. The molecular weights of the major finger print compounds are 197.4, 187.3, and 177.5, respectively.

### Example 6

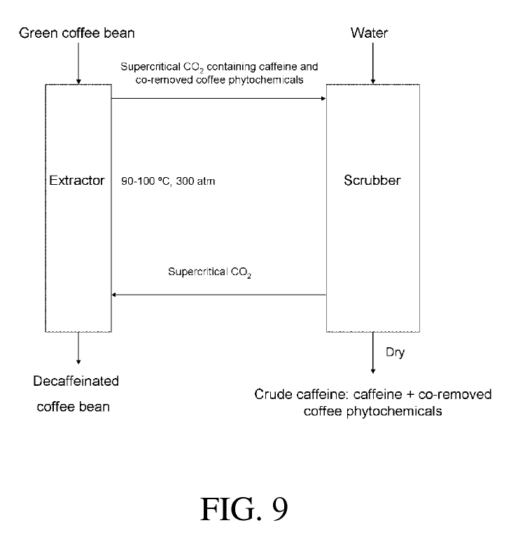

Crude Caffeine. Crude caffeine is available commercially. It is a byproduct of the decaffeination process. In general, the decaffeination process involves extracting raw, green coffee beans with a solvent. FIG. 9 illustrates the preparation of crude caffeine as a byproduct of the supercritical carbon dioxide (scCO₂) decaffeination process. In this process, green coffee beans were soaked in water until the moisture content was 50%. Caffeine was removed in an extractor by liquid carbon dioxide at high temperature (90-100° C.) and high pressure (300 atm). The liquid carbon dioxide was re-circulated between the extractor and a scrubber, where caffeine was removed from the liquid carbon dioxide with water. The resulting caffeine-rich aqueous solution was then concentrated by reverse osmosis and vacuum-dried. Proximate composition of the crude caffeine was analyzed by Silliker, Inc. (South Holland, Ill.).

Total phenolics. The total polyphenol content of the crude caffeine was determined according to the method of Singleton, V. L. & J. A. Rossi, Jr., Am. J. Enol. Vitic. 16: 144-158 (1965). One milliliter of chlorogenic acid standard or crude caffeine solution was mixed with 15 mL water and 1.0 mL Folin-Ciocalteu reagent, and then incubated at room temperature for 10 min. After adding 20% sodium carbonate (3.0 mL) and incubating at 40° C. for 20 min, absorbance was measured at 755 nm with an Agilent 8453 UV-visible spectrophotometer (Waldbronn, Germany). The total polyphenol content was expressed in milligrams of chlorogenic acid equivalents per gram of crude caffeine (CE/g).

Oxygen radical absorbance capacity. To determine antioxidant values of the hydrophilic fraction (ORAC_{hydro}), 5 g crude caffeine was extracted with 20 mL acetone/water (50:50 v/v) on an orbital shaker at room temperature for 1 h. The mixtures were centrifuged at 1972×g in a Rotanta 460R centrifuge (GMI, Ramsey, Minn.). The ORAC_{hydm} values of the supernatants were determined with a method adapted from Ou (Ou, B. et al., Journal of Agricultural and Food Chemistry 49: 4619-4626 (2001)) on a FL600 plate fluorescence reader (Bio-Tek Instruments, Inc., Winooski, Vt.) controlled by KC4 3.0 software. The excitation wavelength was set at 485 (±20) nm and the emission wavelength set at 530 (±25) nm. To determine antioxidant values of the lipophilic fraction (ORACₗᵢₚₒ), 5 g crude caffeine was extracted twice with 10 mL hexane/dichloromethane (50:50 v/v). ORACₗᵢₚₒ values of the combined organic phase were determined according to a previously published method (Wu, X. et al., Journal of Agricultural and Food Chemistry 52 4026-4037 (2004); Huang, D. et al., Journal of Agricultural and Food Chemistry 50: 1815-1821 (2002)).

Glucose uptake. The in vitro effects of crude caffeine on glucose uptake were assessed with human adipocytes and skeletal muscle cells (Zen-Bio, Research Triangle Park, N.C.). Briefly, primary human subcutaneous adipocytes or primary human myoblasts were differentiated in 96-well microplates. The resulting adipocytes (2 weeks post-differentiation) or skeletal muscle cells (10 days post-differentiation) were treated with crude caffeine (0.001-0.5 mg/mL, final concentration) in the presence of ³H-2-deoxyglucose; cells treated with insulin (100 nM) were used as the positive control, and untreated cells as the negative control. All samples, including the positive and negative controls, were tested in triplicate. After a 2-h incubation at 37° C. and 5% CO₂, the cells were washed with PBS and lysed. The cell lysate was mixed with scintillation fluid and radioactivity of each well was measured as counts per minute (CPM).

COX-2 inhibition. Oxygen consumption was measured in the reaction chamber of an Oxytherm (Hansatech Instrumental, Norfolk, England) at 37° C. The reaction mixture consisting of 0.5 mL Tris buffer (0.1 M, pH 8.0), 5 μL heme [100 μM in dimethyl sulfoxide (DMSO)], and 10 μL of COX-2 enzyme was incubated for 1 min. Five microliters of each sample (in DMSO or ethanol) was added and the mixture was incubated for an additional 1 min. The assay was initiated by adding 5 μL arachidonic acid, and the oxygen concentration was monitored. The initial oxygen consumption rate was obtained from the kinetic curve. COX-2 inhibition was expressed as the inhibitor concentration at which the initial oxygen consumption rate decreased by 50% (IC₅₀).

Additive effects of crude caffeine bioactive compounds combined with alpha-tocopherol. In a 15-mL test tube, methyl linoleate (1.2 g), Tween-20 (2.9 g), n-butanol (1.5 g), and potassium phosphate buffer (4.4 g) were vortexed until the resulting solution was clear. An OBS microplate was prepared with duplicate wells of Trolox solution standards (20 μL; 500, 250, 125, 62.5, or 31.25 μM) and triplicate wells of samples diluted in phosphate buffer (20 μL). The perimeter wells were not used for kinetic studies to avoid potential edge effects. The oxidation substrate, methyl linoleate microemulsion (200 μL), was then added. The plate was incubated at 37° C. for 10 min before adding 20 μL 2,2′-azobis(2-amidinopropane)dihydrochloride (AAPH, 0.20 g/mL in phosphate buffer) to each well; the final volume in each well was 240 μL. A control well containing 200 μL of microemulsion and 40 μL of phosphate buffer was used to normalize the fluorescence reading. The reaction kinetics were monitored at 37° C. for 2 h, with readings every 2 min, by a Synergy HT microplate fluorescent reader (Biotek Instruments Inc.). The microplate reader was fitted with an excitation filter at 485 nm, an emission filter of 590 nm, and an oxygen sensor biosystem, and the plate was shaken for 20 s at low intensity before each reading to ensure sufficient mixing. After normalization, the fluorescence data were converted to the oxygen concentration at each point of the reaction. The ORAC values of samples were determined as previously described (Sim, W. L. S. et al., Journal of Agricultural and Food Chemistry 57: 3409-3414 (2009)).

Statistical analysis. Data were analyzed by one-way analysis of variance (ANOVA) followed by Tukey's post hoc comparison test using PASW Statistics 18 (Chicago, Ill.). Results are expressed as mean±standard deviation (SD). P<0.05 was considered statistically significant.

These examples and embodiments are illustrative and are not to be read as limiting the scope of the invention as it is defined by this specification and the appended claims.

All references cited in this specification are incorporated herein by reference.

1. A comestible comprising a comestible base with an effective amount of a functional additive blended therein, the functional additive including a crude caffeine complex of caffeine and effective amounts of coffee-based bioactive components to provide one of stimulate glucose uptake in cells similar to or better than insulin, hydrophilic and lipophilic antioxidant activities, anti-inflammatory activities, and combinations thereof.

2. The comestible of claim 1, wherein the crude caffeine complex includes about 90 to about 95 percent caffeine and about 5 to about 10 percent of the coffee-based bioactive components.

3. The comestible of claim 2 wherein the coffee-based bioactive components are co-eluted with the caffeine in a decaffeination of unroasted, green coffee beans and wherein the bioactive components have molecular weights selected from about 197, about 187, about 177, and mixtures thereof.

4. The comestible of claim 3, wherein the coffee-based bioactive component of the crude caffeine complex includes about 0 to about 20 percent each of the compounds co-eluted with caffeine and having a molecular weight selected from about 197, about 187, about 177, and mixtures thereof.

5. The comestible of claim 1, wherein the comestible includes about 0.05 to about 25 percent of the crude caffeine complex.

6. The comestible product of claim 1, wherein the amount of crude caffeine increases glucose uptake into human skeletal muscle cells at a level statistically similar to 100 nM of insulin.

7. The comestible product of claim 1, wherein the amount of crude caffeine increases glucose uptake into human adipocytes statistically similar to or higher than 100 nM of insulin.

8. The comestible product of claim 1, wherein the comestible base is selected from soft drinks, snacks, cookies, cheese, crackers, powdered beverages, roast and ground coffee, and soluble coffee.

9. The comestible product of claim 1, wherein the crude caffeine complex includes less than about 0.01 percent chlorogenic acids and its lactone derivatives, less than about 0.1 percent sugars, and less than about 0.01 percent proteins.

10. The comestible product of claim 1, wherein the crude caffeine complex is obtained from a supercritical carbon dioxide decaffeination of unroasted, green coffee beans.

11. A method of producing an improved comestible including a crude caffeine complex, the method comprising:
a. decaffeinating unroasted, green coffee beans to produce decaffeinated coffee beans and a crude caffeine extract;
b. recovering the crude caffeine extract;
c. converting the crude caffeine extract into a crude caffeine complex having a major amount of caffeine and a minor amount of coffee-based bioactive compounds; and
d. adding the crude caffeine complex to a comestible in amounts effective to achieve one of stimulated glucose uptake into cells, anti-oxidant properties, and anti-inflammatory activities.

12. The method of claim 11, wherein the crude caffeine complex is about 90 to about 95 percent caffeine and about 5 to about 10 coffee-based bioactive compounds.

13. The method of claim 11, wherein the decaffeination is a supercritical carbon dioxide decaffeination.

14-34. (canceled)

35. The comestible of claim 1, wherein said crude caffeine has an ORAC_{hydro} value of at least about 150 to about 300 μmole TE/g and an ORACiᵢₚₒ value of at least about 50 to about 100 μmole TE/g.

36. The comestible of claim 1, wherein said crude caffeine inhibits COX-2 activity at a level statistically similar to or greater than aspirin.

37-43. (canceled)
